(19) United States
(12) Patent Application Publication
(10) Pub. No.: US 2023/0149515 A1
(43) Pub. Date: May 18, 2023

(54) INHIBITION OF SARS-COV-2 VIRAL ENTRY THROUGH ORAL ADMINISTRATION OF LACTOFERRIN AND USES THEREOF

(71) Applicant: The Regents of the University of Michigan, Ann Arbor, MI (US)

(72) Inventors: Kevin J. Weatherwax, Ann Arbor, MI (US); Carmen Mirabelli, Ann Arbor, MI (US); Jonathan Zachary Sexton, Ann Arbor, MI (US); Sean Michael McCarty, Ann Arbor, MI (US); Jesse Wotring, Ann Arbor, MI (US)

(21) Appl. No.: 17/920,698

(22) PCT Filed: Apr. 29, 2021
(86) PCT No.: PCT/US2021/029917

§ 371 Date: Oct. 21, 2022

Related U.S. Application Data
(60) Provisional application No. 63/017,433, filed on Apr. 29, 2020.
(60) Provisional application No. 63/017,435, filed on Apr. 29, 2020.
(60) Provisional application No. 63/017,436, filed on Apr. 29, 2020.

(51) Int. Cl. A61K 38/40; A61P 31/14; A61K 31/675

(52) U.S. Cl. CPC A61K 38/40; A61P 31/14; A61K 31/675

(57) ABSTRACT

This invention is in the field of medicinal pharmacology. In particular, the present invention relates to pharmaceutical compositions comprising lactoferrin (e.g., naturally occurring lactoferrin, recombinant lactoferrin) which function as inhibitors of severe acute respiratory syndrome coronavirus 2 (SARS-CoV-2) viral entry into a subject (e.g., human subject). The invention further relates to methods of preventing, treating and/or ameliorating symptoms related to conditions caused by the SARS-CoV-2 virus (e.g., COVID-19), comprising administering to a subject (e.g., human subject) lactoferrin (e.g., naturally occurring lactoferrin, recombinant lactoferrin) (e.g., oral formulation of lactoferrin, intravenous (IV) formulation of lactoferrin) alone or with additional agents (e.g., remdesivir).

### CROSS REFERENCE TO RELATED APPLICATIONS

This application claims the benefit of U.S. Prov. Appl. 63/017,433 filed Apr. 29, 2020, U.S. Prov. Appl. 63/017,435 filed Apr. 29, 2020, and U.S. Prov. Appl. 63/017,436 filed Apr. 29, 2020, the entire contents of which are incorporated herein by reference.

### FIELD OF THE INVENTION

This invention is in the field of medicinal pharmacology. In particular, the present invention relates to pharmaceutical compositions comprising lactoferrin (e.g., naturally occurring lactoferrin, recombinant lactoferrin) which function as inhibitors of severe acute respiratory syndrome coronavirus 2 (SARS-CoV-2) viral entry into a subject (e.g., human subject). The invention further relates to methods of preventing, treating and/or ameliorating symptoms related to conditions caused by the SARS-CoV-2 virus (e.g., COVID-19), comprising administering to a subject (e.g., human subject) lactoferrin (e.g., naturally occurring lactoferrin, recombinant lactoferrin) (e.g., oral formulation of lactoferrin, intravenous (IV) formulation of lactoferrin) alone or with additional agents (e.g., remdesivir).

### INTRODUCTION

The global pandemic of COVID-19 caused by severe acute respiratory syndrome coronavirus 2 (SARS-CoV-2) began in Wuhan, China, in December 2019, and has since spread worldwide (see, Zhu N, et al., N Engl J Med. 2020;382(8):727-733). As of Apr. 5, 2020, there have been more than 1.2 million reported cases and 69 000 deaths in more than 200 countries. This novel Betacoronavirus is similar to severe acute respiratory syndrome coronavirus (SARS-CoV) and Middle East respiratory syndrome coronavirus (MERS-CoV); based on its genetic proximity, it likely originated from bat-derived coronaviruses with spread via an unknown intermediate mammal host to humans (see, Zhu N, et al., N Engl J Med. 2020;382(8):727-733). The viral genome of SARS-CoV-2 was rapidly sequenced to enable diagnostic testing, epidemiologic tracking, and development of preventive and therapeutic strategies.

Improved techniques for preventing, treating and ameliorating the symptoms of SARS-CoV-2 are desperately needed.

The present invention addresses this need.

### SUMMARY

Lactoferrin is a single chain metal binding glycoprotein. Many cells types, such as monocytes, macrophages, lymphocytes, and brush-border cells, are known to have lactoferrin receptors. In addition to lactoferrin being an essential growth factor for both B and T lymphocytes, lactoferrin has a wide array of functions related to host primary defense mechanisms. For example, lactoferrin has been reported to activate natural killer (NK) cells, induce colony stimulating activity, activate polymorphonuclear neutrophils (PMN), regulate granulopoeisis, enhance antibody-dependent cell cytotoxicity, stimulate lymphokine-activated killer (LAK) cell activity, and potentiate macrophage toxicity.

There is strong biological plausibility for an anti-viral effect of lactoferrin on SARS-CoV-2. Lactoferrin gene expression has been shown to be highly upregulated in response to SARS (Reghunathan et al, BMC Immunology, 2005) and, in addition to enhancing NK cell and neutrophil activity, blocks SARS-CoV entry through binding to heparin sulfate proteoglycans (Lang let al, PLOS One, 2011). Heparin sulfate proteoglycans are thought to be important binding sites for coronavirus in the early attachment phase. Lactoferrin is thought to inhibit Dengue virus and Toscana virus infectivity via a similar mechanism (Chen et al, Int J Mol Sci, 2017; Pietrantoni et al, Viruses, 2015). Lactoferrin has broad anti-viral activity, including against cytomegalovirus and human immunodeficiency virus (Harmsen et al, J Infect Dis, 1995); hepatitis C (Ikeda et al, Biochem Biphys Res Comm, 1998); Chikungunya and Zika viruses (Carvalho et al, J Gen Virology, 2017); Influenza A (Superti et al, Viruses, 2019); and others.

With respect to COVID-19, orally available lactoferrin could be especially effective in mitigating gastrointestinal symptoms. For example, lactoferrin dysregulation or relative depletion during SARS-CoV-2 infection could mediate COVID-related diarrhea; this is supported by the association of lactoferrin gene polymorphisms and increased susceptibility to infectious diarrhea (Mohamed et al, Clin Infect Dis, 2007). Additionally, lactoferrin is a strong suppressor of several pro-inflammatory cytokines including IL-6 (Actor et al, Cur Pharm Des, 2009), which is highly overexpressed in SARS-Cov-2 patients and contributes to an associated “cytokine storm” (Conti et al, J Biol Regul Homeost Agents, 2020). Indeed, the immunomodulatory effect of lactoferrin could be very useful for the treatment of SARS-CoV-2 patients.

With the emergence of the Covid-19 pandemic, rapidly deployable interventions are critically needed to reduce the morbidity and mortality of the disease. The Michigan Center for Drug Repurposing (CDR) has a fully developed high-content screening pipeline currently being used to screen approved drugs against the causative agent, SARS Cov-2. The CDR has an approved Biosafety Level 3 (BSL3) protocol, supporting screening of individual approved drugs followed by high content imaging to characterize the mechanism of action of effective drugs.

Experiments implemented during the course of developing embodiments for the present invention conducted a high throughput screen of FDA-approved drugs for efficacy in a SARS-CoV-2 infectivity assay. This high content bioimaging-based assay has proven to be highly robust and informative and has already led to the identification of FDA approved drugs that can be rapidly translated into clinical use. This high-content screen identifies individual agents effective in reducing viral infectivity or replication by imaging individual SARS Cov-2 infected cells. However, because these drugs have not been optimized for anti-Covid19 efficacy, it was hypothesized that combining synergistic drugs representing multiple mechanisms of action into antiviral cocktails could deliver a highly efficacious therapy for Covid19. This strategy has proven highly effective in other viral diseases like HIV and Hepatitis C, where 3 or 4 drug cocktails result in remission of symptoms and undetectable viral load in the patient. Candidate cocktails are evaluated in this high throughput assay with the goal of detecting synergistic efficacy.

Such experiments resulted in a completed screening of 1,200 compounds through the validated assay platform and resulted in the selection of Remdesivir, Gilead's viral replication inhibitor, as a positive control which resulted in 100% efficacy (see, FIG. 1). Such experiments further demonstrated that lactoferrin showed nearly 100% efficacy in preventing SARS-CoV-2 infection in Huh-7 cells as shown in FIG. 2. Moreover, a dose response study of lactoferrin showed that there was over a 95% reduction in SARS-Cov-2 synctitia at all concentrations tested ranging from 200 ug/mL to 12.5 ug/mL. The characteristic reduction is synctitia, as well as the efficacy of lactoferrin are shown in FIG. 3. FIG. 4 shows the amount of infected cells upon exposure to a combination of remesivir and lactoferrin in comparison to only remdesivir. As shown, the combination of remdesivir and lactoferrin demonstrated increased anti-viral activity in comparison to only administering remdesivir.

Accordingly, the present invention relates to pharmaceutical compositions comprising lactoferrin (e.g., naturally occurring lactoferrin, recombinant lactoferrin) (e.g., oral formulation of lactoferrin, intravenous (IV) formulation of lactoferrin) alone or with additional agents (e.g., hydroxychloroquine, dexamethasone, and remdesivir) which function as inhibitors of severe acute respiratory syndrome coronavirus 2 (SARS-CoV-2) viral entry into a subject (e.g., human subject). The invention further relates to methods of preventing, treating and/or ameliorating symptoms related to conditions caused by the SARS-CoV-2 virus (e.g., COVID-19), comprising administering to a subject (e.g., a human patient) a pharmaceutical composition comprising lactoferrin (e.g., naturally occurring lactoferrin, recombinant lactoferrin) (e.g., oral formulation of lactoferrin, intravenous (IV) formulation of lactoferrin) alone or with additional agents (e.g., hydroxychloroquine, dexamethasone, and remdesivir).

In certain embodiments, the present invention provides methods for administering a pharmaceutical composition comprising lactoferrin (e.g., naturally occurring lactoferrin, recombinant lactoferrin) (e.g., oral formulation of lactoferrin, intravenous (IV) formulation of lactoferrin) alone or with additional agents (e.g., remdesivir) to a subject (e.g., a human subject) (e.g., a human subject suffering from or at risk of suffering from a condition related to SARS-CoV-2 infection (e.g., COVID-19)) for purposes of treating, preventing and/or ameliorating the symtpoms of a viral infection (e.g., SARS-CoV-2 infection (e.g., COVID-19)).

In such embodiments, the methods are not limited treating, preventing and/or ameliorating the symtpoms of a particular type or kind of viral infection. In some embodiments, the viral infection is a SARS-CoV-2 related viral infection (e.g., COVID-19). In some embodiments, the viral infection is any infection related to influenza, HIV, HIV-1, HIV-2, drug-resistant HIV, Junin virus, Chikungunya virus, Yellow Fever virus, Dengue virus, Pichinde virus, Lassa virus, adenovirus, Measles virus, Punta Toro virus, Respiratory Syncytial virus, Rift Valley virus, RHDV, SARS coronavirus, Tacaribe virus, and West Nile virus. In some embodiments, the viral infection is associated with any virals having M^{pro} protease activity and/or expression.

In such embodiments, administration of the pharmaceutical composition comprising lactoferrin (e.g., naturally occurring lactoferrin, recombinant lactoferrin) (e.g., oral formulation of lactoferrin, intravenous (IV) formulation of lactoferrin) alone or with additional agents (e.g., hydroxychloroquine, dexamethasone, and remdesivir) results in suppression of pro-inflammatory cytokine activity (e.g., IL-6 activity) within the subject. In some embodiments, administration of the pharmaceutical composition results in enhancement of NK cell activity within the subject. In some embodiments, administration of the pharmaceutical composition results in enhancement of neutrophil activity within the subject. In some embodiments, administration of the pharmaceutical composition results in inhibition of viral entry into the subject's cells through inhibiting binding of the virus with heparin sulfate proteoglycan within such cells.

In certain embodiments, the present invention provides methods for treating, ameliorating and/or preventing a condition related to viral infection in a subject, comprising administering to the subject a pharmaceutical composition comprising lactoferrin (e.g., naturally occurring lactoferrin, recombinant lactoferrin) (e.g., oral formulation of lactoferrin, intravenous (IV) formulation of lactoferrin) alone or with additional agents (e.g., remdesivir). In some embodiments, the subject is a human subject. In some embodiments, the subject is a human subject suffering from or at risk of suffering from a condition related to SARS-CoV-2 infection (e.g., COVID-19). In some embodiments, the viral infection is a SARS-CoV-2 viral infection.

In certain embodiments, the present invention provides methods for treating, ameliorating and/or preventing SARS-CoV-2 infection (e.g., COVID-19) in a subject, comprising administering to the subject a pharmaceutical composition lactoferrin (e.g., naturally occurring lactoferrin, recombinant lactoferrin) (e.g., oral formulation of lactoferrin, intravenous (IV) formulation of lactoferrin) alone or with additional agents (e.g., remdesivir). In some embodiments, the subject is a human subject.

In certain embodiments, the present invention provides methods for treating, ameliorating and/or preventing symptoms related to viral infection in a subject, comprising administering to the subject a pharmaceutical composition comprising lactoferrin (e.g., naturally occurring lactoferrin, recombinant lactoferrin) (e.g., oral formulation of lactoferrin, intravenous (IV) formulation of lactoferrin) alone or with additional agents (e.g., remdesivir). In some embodiments, the subject is a human subject. In some embodiments, the subject is a human subject suffering from or at risk of suffering from a condition related to SARS-CoV-2 infection (e.g., COVID-19). In some embodiments, the subject is a human subject suffering from a SARS-CoV-2 viral infection. In some embodiments, the one or more symptoms related to viral infection includes, but is not limited to, fever, fatigue, dry cough, myalgias, dyspnea, acute respiratory distress syndrome, and pneumonia.

In certain embodiments, the present invention provides methods for treating, ameliorating and/or preventing symptoms related to SARS-CoV-2 infection (e.g., COVID-19) in a subject, comprising lactoferrin (e.g., lactoferrin formulated for oral administration, IV formulation of lactoferrin, recombinant lactoferrin (e.g., IV formulation of recombinant lactoferrin) alone or with additional agents (e.g., hydroxychloroquine, dexamethasone, and remdesivir). In some embodiments, the subject is a human subject. In some embodiments, the one or more symptoms related to viral infection includes, but is not limited to, fever, fatigue, dry cough, myalgias, dyspnea, acute respiratory distress syndrome, and pneumonia.

In certain embodiments, the present invention provides methods for treating, ameliorating and/or preventing acute respiratory distress syndrome in a subject, comprising administering to the subject a pharmaceutical composition comprising lactoferrin (e.g., naturally occurring lactoferrin, recombinant lactoferrin) (e.g., oral formulation of lactoferrin, intravenous (IV) formulation of lactoferrin) alone or with additional agents (e.g., remdesivir). In some embodiments, the subject is a human subject. In some embodiments, the subject is a human subject suffering from or at risk of suffering from a condition related to SARS-CoV-2 infection (e.g., COVID-19). In some embodiments, the subject is a human subject suffering from a SARS-CoV-2 viral infection.

In certain embodiments, the present invention provides methods for treating, ameliorating and/or preventing acute respiratory distress syndrome related to SARS-CoV-2 infection (e.g., COVID-19) in a subject, comprising administering to the subject a pharmaceutical composition comprising lactoferrin (e.g., naturally occurring lactoferrin, recombinant lactoferrin) (e.g., oral formulation of lactoferrin, intravenous (IV) formulation of lactoferrin) alone or with additional agents (e.g., remdesivir). In some embodiments, the subject is a human subject. In some embodiments, the subject is a human subject suffering from or at risk of suffering from a condition related to SARS-CoV-2 infection (e.g., COVID-19). In some embodiments, the subject is a human subject suffering from a SARS-CoV-2 viral infection.

In certain embodiments, the present invention provides methods for treating, ameliorating and/or preventing pneumonia in a subject, comprising administering to the subject a pharmaceutical composition comprising lactoferrin (e.g., naturally occurring lactoferrin, recombinant lactoferrin) (e.g., oral formulation of lactoferrin, intravenous (IV) formulation of lactoferrin) alone or with additional agents (e.g., remdesivir). In some embodiments, the subject is a human subject. In some embodiments, the subject is a human subject suffering from or at risk of suffering from a condition related to SARS-CoV-2 infection (e.g., COVID-19). In some embodiments, the subject is a human subject suffering from a SARS-CoV-2 viral infection.

In certain embodiments, the present invention provides methods for treating, ameliorating and/or preventing pneumonia related to SARS-CoV-2 infection (e.g., COVID-19) in a subject, comprising administering to the subject a pharmaceutical composition comprising lactoferrin (e.g., naturally occurring lactoferrin, recombinant lactoferrin) (e.g., oral formulation of lactoferrin, intravenous (IV) formulation of lactoferrin) alone or with additional agents (e.g., hydroxychloroquine, dexamethasone, and remdesivir). In some embodiments, the subject is a human subject. In some embodiments, the subject is a human subject suffering from or at risk of suffering from a condition related to SARS-CoV-2 infection (e.g., COVID-19). In some embodiments, the subject is a human subject suffering from a SARS-CoV-2 viral infection.

In some embodiments involving the treatment of acute respiratory distress syndrome and/or pneumoina, the pharmaceutical composition comprising lactoferrin (e.g., naturally occurring lactoferrin, recombinant lactoferrin) (e.g., oral formulation of lactoferrin, intravenous (IV) formulation of lactoferrin) alone or with additional agents (e.g., remdesivir) is administered in combination with a known agent to treat respiratory diseases. Known or standard agents or therapies that are used to treat respiratory diseases include, anti-asthma agent/therapies, anti-rhinitis agents/therapies, anti-sinusitis agents/therapies, anti-emphysema agents/therapies, anti-bronchitis agents/therapies or anti-chronic obstructive pulmonary disease agents/therapies. Anti-asthma agents/therapies include mast cell degranulation agents, leukotriene inhibitors, corticosteroids, beta-antagonists, IgE binding inhibitors, anti-CD23 antibody, tryptase inhibitors, and VIP agonists. Anti-allergic rhinitis agents/therapies include H1 antihistamines, alpha-adrenergic agents, and glucocorticoids. Anti-chronic sinusitis therapies include, but are not limited to surgery, corticosteroids, antibiotics, anti-fungal agents, salt-water nasal washes or sprays, anti-inflammatory agents, decongestants, guaifensesin, potassium iodide, luekotriene inhibitors, mast cell degranulating agents, topical moisterizing agents, hot air inhalation, mechanical breathing devices, enzymatic cleaners and antihistamine sprays. Anti-emphysema, anti-bronchitis or anti-chronic obstructive pulmonary disease agents/therapies include, but are not limited to oxygen, bronchodilator agents, mycolytic agents, steroids, antibiotics, anti-fungals, moisterization by nebulization, anti-tussives, respiratory stimulants, surgery and alpha 1 antitrypsin.

In certain embodiments, the present invention provides methods for treating, ameliorating and/or preventing gastrointestinal conditions in a subject, comprising administering to the subject a pharmaceutical composition comprising lactoferrin (e.g., naturally occurring lactoferrin, recombinant lactoferrin) (e.g., oral formulation of lactoferrin, intravenous (IV) formulation of lactoferrin) alone or with additional agents (e.g., remdesivir). In some embodiments, the subject is a human subject. In some embodiments, the subject is a human subject suffering from or at risk of suffering from a condition related to SARS-CoV-2 infection (e.g., COVID-19). In some embodiments, the subject is a human subject suffering from a SARS-CoV-2 viral infection. In some embodiments, the gastrointestinal condition is diarrhea. In some embodiments, the gastrointestinal condition is selected from constipation, irritable bowel syndrome, hemorrhoids, anal fissures, perianal abscesses, anal fistulas, perianal infections, diverticular diseases, colitis, and diarrhea.

In certain embodiments, the present invention provides methods for treating, ameliorating and/or preventing gastrointestinal conditions related to SARS-CoV-2 infection (e.g., COVID-19) in a subject, comprising administering to the subject a pharmaceutical composition comprising lactoferrin (e.g., naturally occurring lactoferrin, recombinant lactoferrin) (e.g., oral formulation of lactoferrin, intravenous (IV) formulation of lactoferrin) alone or with additional agents (e.g., remdesivir). In some embodiments, the subject is a human subject. In some embodiments, the subject is a human subject suffering from or at risk of suffering from a condition related to SARS-CoV-2 infection (e.g., COVID-19). In some embodiments, the subject is a human subject suffering from a SARS-CoV-2 viral infection. In some embodiments, the gastrointestinal condition is diarrhea. In some embodiments, the gastrointestinal condition is selected from constipation, irritable bowel syndrome, hemorrhoids, anal fissures, perianal abscesses, anal fistulas, perianal infections, diverticular diseases, colitis, and diarrhea.

In certain embodiments, the present invention provides methods for inhibiting viral entry in a cell, comprising exposing the cell a pharmaceutical composition comprising lactoferrin (e.g., naturally occurring lactoferrin, recombinant lactoferrin) (e.g., oral formulation of lactoferrin, intravenous (IV) formulation of lactoferrin) alone or with additional agents (e.g., remdesivir). In some embodiments, the cell is at risk of viral infection (e.g., a cell at risk of SARS-CoV-2 infection). In some embodiments, the cell has been exposed to a virus (e.g., a cell currently exposed to SARS-CoV-2). In some embodiments, the cell is in culture. In some embodiments, the cell is a living cell in a subject (e.g., a human subject) (e.g., a human subject suffering from COVID-19) (e.g., a human subject at risk of suffering from COVID-19). In some embodiments, exposure of the cell to the pharmaceutical composition comprising lactoferrin (e.g., naturally occurring lactoferrin, recombinant lactoferrin) (e.g., oral formulation of lactoferrin, intravenous (IV) formulation of lactoferrin) alone or with additional agents (e.g., remdesivir) results in suppression of pro-inflammatory cytokine activity (e.g., IL-6 activity) within the cell. In some embodiments, exposure of the cell to the pharmaceutical composition results in enhancement of NK cell activity within the cell. In some embodiments, exposure of the cell to the pharmaceutical composition results in enhancement of neutrophil activity within the cell. In some embodiments, exposure of the cell to the pharmaceutical composition results in inhibition of viral entry into the cell through inhibiting binding of the virus with heparin sulfate proteoglycan within the cell.

In any of the embodiments described herein, the lactoferrin is naturally occurring lactoferrin. In any of the embodiments described herein, the lactoferrin is recombinant lactoferrin. In some embodiments, the lactoferrin is human lactoferrin produced recombinantly using genetic engineering techniques well known and used in the art, such as recombinant expression or direct production in genetically altered animals, plants or eukaryotes, or chemical synthesis (see, U.S. Pat. Nos. 5,571,896; 5,571,697 and 5,571,691).

Lactoferrin for use in the present disclosure may be, for example, isolated from the milk of a non-human animal or produced by a genetically modified organism. The compositions comprising lactoferrin described herein can, in some embodiments, comprise non-human lactoferrin, non-human lactoferrin produced by a genetically modified organism and/or human lactoferrin produced by a genetically modified organism. Suitable non-human lactoferrins for use in the present disclosure include, but are not limited to, those having at least 48% homology with the amino acid sequence of human lactoferrin. For instance, bovine lactoferrin has an amino acid composition which has about 70% sequence homology to that of human lactoferrin. In some embodiments, the non-human lactoferrin has at least 65% homology with human lactoferrin and in some embodiments, at least 75% homology. Non-human lactoferrins acceptable for use in the present disclosure include, without limitation, bovine lactoferrin, porcine lactoferrin, equine lactoferrin, buffalo lactoferrin, goat lactoferrin, murine lactoferrin and camel lactoferrin.

In certain embodiments, the present invention provides kits comprising (1) a pharmaceutical composition comprising lactoferrin (e.g., naturally occurring lactoferrin, recombinant lactoferrin) (e.g., oral formulation of lactoferrin, intravenous (IV) formulation of lactoferrin) alone or with additional agents (e.g., hydroxychloroquine, dexamethasone, and remdesivir), (2) a container, pack, or dispenser, and (3) instructions for administration.

In certain embodiments, the present invention provides methods for treating, ameliorating and/or preventing a condition related to viral infection in a subject, comprising administering to the subject a pharmaceutical composition comprising lactoferrin. In some embodiments, the pharmaceutical composition comprising lactoferrin is configured for oral administration. In some embodiments, the subject is a human subject. In some embodiments, the subject is a human subject suffering from or at risk of suffering from a condition related to SARS-CoV-2 infection (e.g., COVID-19). In some embodiments, the viral infection is a SARS-CoV-2 viral infection.

In certain embodiments, the present invention provides methods for treating, ameliorating and/or preventing SARS-CoV-2 infection (e.g., COVID-19) in a subject, comprising administering to the subject a pharmaceutical composition comprising lactoferrin. In some embodiments, the pharmaceutical composition comprising lactoferrin is configured for oral administration. In some embodiments, the subject is a human subject.

In certain embodiments, the present invention provides methods for treating, ameliorating and/or preventing symptoms related to viral infection in a subject, comprising administering to the subject a pharmaceutical composition comprising lactoferrin. In some embodiments, the pharmaceutical composition comprising lactoferrin is configured for oral administration. In some embodiments, the subject is a human subject. In some embodiments, the subject is a human subject suffering from or at risk of suffering from a condition related to SARS-CoV-2 infection (e.g., COVID-19). In some embodiments, the subject is a human subject suffering from a SARS-CoV-2 viral infection. In some embodiments, the one or more symptoms related to viral infection includes, but is not limited to, fever, fatigue, dry cough, myalgias, dyspnea, acute respiratory distress syndrome, and pneumonia.

In certain embodiments, the present invention provides methods for treating, ameliorating and/or preventing symptoms related to SARS-CoV-2 infection (e.g., COVID-19) in a subject, comprising administering to the subject a pharmaceutical composition comprising lactoferrin. In some embodiments, the pharmaceutical composition comprising lactoferrin is configured for oral administration. In some embodiments, the subject is a human subject. In some embodiments, the one or more symptoms related to viral infection includes, but is not limited to, fever, fatigue, dry cough, myalgias, dyspnea, acute respiratory distress syndrome, and pneumonia.

In certain embodiments, the present invention provides methods for treating, ameliorating and/or preventing acute respiratory distress syndrome in a subject, comprising administering to the subject a pharmaceutical composition comprising lactoferrin. In some embodiments, the pharmaceutical composition comprising lactoferrin is configured for oral administration. In some embodiments, the subject is a human subject. In some embodiments, the subject is a human subject suffering from or at risk of suffering from a condition related to SARS-CoV-2 infection (e.g., COVID-19). In some embodiments, the subject is a human subject suffering from a SARS-CoV-2 viral infection.

In certain embodiments, the present invention provides methods for treating, ameliorating and/or preventing acute respiratory distress syndrome related to SARS-CoV-2 infection (e.g., COVID-19) in a subject, comprising administering to the subject a pharmaceutical composition comprising lactoferrin. In some embodiments, the pharmaceutical composition comprising lactoferrin is configured for oral administration. In some embodiments, the subject is a human subject. In some embodiments, the subject is a human subject suffering from or at risk of suffering from a condition related to SARS-CoV-2 infection (e.g., COVID-19). In some embodiments, the subject is a human subject suffering from a SARS-CoV-2 viral infection.

In certain embodiments, the present invention provides methods for treating, ameliorating and/or preventing pneumonia in a subject, comprising administering to the subject a pharmaceutical composition comprising lactoferrin. In some embodiments, the pharmaceutical composition comprising lactoferrin is configured for oral administration. In some embodiments, the subject is a human subject. In some embodiments, the subject is a human subject suffering from or at risk of suffering from a condition related to SARS-CoV-2 infection (e.g., COVID-19). In some embodiments, the subject is a human subject suffering from a SARS-CoV-2 viral infection.

In certain embodiments, the present invention provides methods for treating, ameliorating and/or preventing pneumonia related to SARS-CoV-2 infection (e.g., COVID-19) in a subject, comprising administering to the subject a pharmaceutical composition comprising lactoferrin. In some embodiments, the pharmaceutical composition comprising lactoferrin is configured for oral administration. In some embodiments, the subject is a human subject. In some embodiments, the subject is a human subject suffering from or at risk of suffering from a condition related to SARS-CoV-2 infection (e.g., COVID-19). In some embodiments, the subject is a human subject suffering from a SARS-CoV-2 viral infection.

In some embodiments involving the treatment of acute respiratory distress syndrome and/or pneumoina, the pharmaceutical composition comprising lactoferrin is administered in combination with a known agent to treat respiratory diseases. Known or standard agents or therapies that are used to treat respiratory diseases include, anti-asthma agent/therapies, anti-rhinitis agents/therapies, anti-sinusitis agents/therapies, anti-emphysema agents/therapies, anti-bronchitis agents/therapies or anti-chronic obstructive pulmonary disease agents/therapies. Anti-asthma agents/therapies include mast cell degranulation agents, leukotriene inhibitors, corticosteroids, beta-antagonists, IgE binding inhibitors, anti-CD23 antibody, tryptase inhibitors, and VIP agonists. Anti-allergic rhinitis agents/therapies include H1 antihistamines, alpha-adrenergic agents, and glucocorticoids. Anti-chronic sinusitis therapies include, but are not limited to surgery, corticosteroids, antibiotics, anti-fungal agents, salt-water nasal washes or sprays, anti-inflammatory agents, decongestants, guaifensesin, potassium iodide, luekotriene inhibitors, mast cell degranulating agents, topical moisterizing agents, hot air inhalation, mechanical breathing devices, enzymatic cleaners and antihistamine sprays. Anti-emphysema, anti-bronchitis or anti-chronic obstructive pulmonary disease agents/therapies include, but are not limited to oxygen, bronchodilator agents, mycolytic agents, steroids, antibiotics, anti-fungals, moisterization by nebulization, anti-tussives, respiratory stimulants, surgery and alpha 1 antitrypsin.

In certain embodiments, the present invention provides methods for treating, ameliorating and/or preventing gastrointestinal conditions in a subject, comprising administering to the subject a pharmaceutical composition comprising lactoferrin. In some embodiments, the pharmaceutical composition comprising lactoferrin is configured for oral administration. In some embodiments, the subject is a human subject. In some embodiments, the subject is a human subject suffering from or at risk of suffering from a condition related to SARS-CoV-2 infection (e.g., COVID-19). In some embodiments, the subject is a human subject suffering from a SARS-CoV-2 viral infection. In some embodiments, the gastrointestinal condition is diarrhea. In some embodiments, the gastrointestinal condition is selected from constipation, irritable bowel syndrome, hemorrhoids, anal fissures, perianal abscesses, anal fistulas, perianal infections, diverticular diseases, colitis, and diarrhea.

In certain embodiments, the present invention provides methods for treating, ameliorating and/or preventing gastrointestinal conditions related to SARS-CoV-2 infection (e.g., COVID-19) in a subject, comprising administering to the subject a pharmaceutical composition comprising lactoferrin. In some embodiments, the pharmaceutical composition comprising lactoferrin is configured for oral administration. In some embodiments, the subject is a human subject. In some embodiments, the subject is a human subject suffering from or at risk of suffering from a condition related to SARS-CoV-2 infection (e.g., COVID-19). In some embodiments, the subject is a human subject suffering from a SARS-CoV-2 viral infection. In some embodiments, the gastrointestinal condition is diarrhea. In some embodiments, the gastrointestinal condition is selected from constipation, irritable bowel syndrome, hemorrhoids, anal fissures, perianal abscesses, anal fistulas, perianal infections, diverticular diseases, colitis, and diarrhea.

In certain embodiments, the present invention provides methods for inhibiting viral entry in a cell, comprising exposing the cell a pharmaceutical composition comprising lactoferrin. In some embodiments, the cell is at risk of viral infection (e.g., a cell at risk of SARS-CoV-2 infection). In some embodiments, the cell has been exposed to a virus (e.g., a cell currently exposed to SARS-CoV-2). In some embodiments, the cell is in culture. In some embodiments, the cell is a living cell in a subject (e.g., a human subject) (e.g., a human subject suffering from COVID-19) (e.g., a human subject at risk of suffering from COVID-19). In some embodiments, exposure of the cell to the pharmaceutical composition comprising lactoferrin results in suppression of pro-inflammatory cytokine activity (e.g., IL-6 activity) within the cell. In some embodiments, exposure of the cell to the pharmaceutical composition comprising lactoferrin results in enhancement of NK cell activity within the cell. In some embodiments, exposure of the cell to the pharmaceutical composition comprising lactoferrin results in enhancement of neutrophil activity within the cell. In some embodiments, exposure of the cell to the pharmaceutical composition comprising lactoferrin results in inhibition of viral entry into the cell through inhibiting binding of the virus with heparin sulfate proteoglycan within the cell.

In certain embodiments, the present invention provides methods for treating, ameliorating and/or preventing a condition related to viral infection in a subject, comprising administering to the subject a pharmaceutical composition comprising lactoferrin. In some embodiments, the pharmaceutical composition comprising lactoferrin is configured for oral administration. In some embodiments, the subject is a human subject. In some embodiments, the subject is a human subject suffering from or at risk of suffering from a condition related to SARS-CoV-2 infection (e.g., COVID-19). In some embodiments, the viral infection is a SARS-CoV-2 viral infection.

In certain embodiments, the present invention provides methods for treating, ameliorating and/or preventing SARS-CoV-2 infection (e.g., COVID-19) in a subject, comprising administering to the subject a pharmaceutical composition comprising lactoferrin. In some embodiments, the pharmaceutical composition comprising lactoferrin is configured for oral administration. In some embodiments, the subject is a human subject.

In certain embodiments, the present invention provides methods for treating, ameliorating and/or preventing symptoms related to viral infection in a subject, comprising administering to the subject a pharmaceutical composition comprising lactoferrin. In some embodiments, the pharmaceutical composition comprising lactoferrin is configured for oral administration. In some embodiments, the subject is a human subject. In some embodiments, the subject is a human subject suffering from or at risk of suffering from a condition related to SARS-CoV-2 infection (e.g., COVID-19). In some embodiments, the subject is a human subject suffering from a SARS-CoV-2 viral infection. In some embodiments, the one or more symptoms related to viral infection includes, but is not limited to, fever, fatigue, dry cough, myalgias, dyspnea, acute respiratory distress syndrome, and pneumonia.

In certain embodiments, the present invention provides methods for treating, ameliorating and/or preventing symptoms related to SARS-CoV-2 infection (e.g., COVID-19) in a subject, comprising administering to the subject a pharmaceutical composition comprising lactoferrin. In some embodiments, the pharmaceutical composition comprising lactoferrin is configured for oral administration. In some embodiments, the subject is a human subject. In some embodiments, the one or more symptoms related to viral infection includes, but is not limited to, fever, fatigue, dry cough, myalgias, dyspnea, acute respiratory distress syndrome, and pneumonia.

In certain embodiments, the present invention provides methods for treating, ameliorating and/or preventing acute respiratory distress syndrome in a subject, comprising administering to the subject a pharmaceutical composition comprising lactoferrin. In some embodiments, the pharmaceutical composition comprising lactoferrin is configured for oral administration. In some embodiments, the subject is a human subject. In some embodiments, the subject is a human subject suffering from or at risk of suffering from a condition related to SARS-CoV-2 infection (e.g., COVID-19). In some embodiments, the subject is a human subject suffering from a SARS-CoV-2 viral infection.

In certain embodiments, the present invention provides methods for treating, ameliorating and/or preventing acute respiratory distress syndrome related to SARS-CoV-2 infection (e.g., COVID-19) in a subject, comprising administering to the subject a pharmaceutical composition comprising lactoferrin. In some embodiments, the pharmaceutical composition comprising lactoferrin is configured for oral administration. In some embodiments, the subject is a human subject. In some embodiments, the subject is a human subject suffering from or at risk of suffering from a condition related to SARS-CoV-2 infection (e.g., COVID-19). In some embodiments, the subject is a human subject suffering from a SARS-CoV-2 viral infection.

In certain embodiments, the present invention provides methods for treating, ameliorating and/or preventing pneumonia in a subject, comprising administering to the subject a pharmaceutical composition comprising lactoferrin. In some embodiments, the pharmaceutical composition comprising lactoferrin is configured for oral administration. In some embodiments, the subject is a human subject. In some embodiments, the subject is a human subject suffering from or at risk of suffering from a condition related to SARS-CoV-2 infection (e.g., COVID-19). In some embodiments, the subject is a human subject suffering from a SARS-CoV-2 viral infection.

In certain embodiments, the present invention provides methods for treating, ameliorating and/or preventing pneumonia related to SARS-CoV-2 infection (e.g., COVID-19) in a subject, comprising administering to the subject a pharmaceutical composition comprising lactoferrin. In some embodiments, the pharmaceutical composition comprising lactoferrin is configured for oral administration. In some embodiments, the subject is a human subject. In some embodiments, the subject is a human subject suffering from or at risk of suffering from a condition related to SARS-CoV-2 infection (e.g., COVID-19). In some embodiments, the subject is a human subject suffering from a SARS-CoV-2 viral infection.

In some embodiments involving the treatment of acute respiratory distress syndrome and/or pneumoina, the pharmaceutical composition comprising lactoferrin is administered in combination with a known agent to treat respiratory diseases. Known or standard agents or therapies that are used to treat respiratory diseases include, anti-asthma agent/therapies, anti-rhinitis agents/therapies, anti-sinusitis agents/therapies, anti-emphysema agents/therapies, anti-bronchitis agents/therapies or anti-chronic obstructive pulmonary disease agents/therapies. Anti-asthma agents/therapies include mast cell degranulation agents, leukotriene inhibitors, corticosteroids, beta-antagonists, IgE binding inhibitors, anti-CD23 antibody, tryptase inhibitors, and VIP agonists. Anti-allergic rhinitis agents/therapies include H1 antihistamines, alpha-adrenergic agents, and glucocorticoids. Anti-chronic sinusitis therapies include, but are not limited to surgery, corticosteroids, antibiotics, anti-fungal agents, salt-water nasal washes or sprays, anti-inflammatory agents, decongestants, guaifensesin, potassium iodide, luekotriene inhibitors, mast cell degranulating agents, topical moisterizing agents, hot air inhalation, mechanical breathing devices, enzymatic cleaners and antihistamine sprays. Anti-emphysema, anti-bronchitis or anti-chronic obstructive pulmonary disease agents/therapies include, but are not limited to oxygen, bronchodilator agents, mycolytic agents, steroids, antibiotics, anti-fungals, moisterization by nebulization, anti-tussives, respiratory stimulants, surgery and alpha 1 antitrypsin.

In certain embodiments, the present invention provides methods for treating, ameliorating and/or preventing gastrointestinal conditions in a subject, comprising administering to the subject a pharmaceutical composition comprising lactoferrin. In some embodiments, the pharmaceutical composition comprising lactoferrin is configured for oral administration. In some embodiments, the subject is a human subject. In some embodiments, the subject is a human subject suffering from or at risk of suffering from a condition related to SARS-CoV-2 infection (e.g., COVID-19). In some embodiments, the subject is a human subject suffering from a SARS-CoV-2 viral infection. In some embodiments, the gastrointestinal condition is diarrhea. In some embodiments, the gastrointestinal condition is selected from constipation, irritable bowel syndrome, hemorrhoids, anal fissures, perianal abscesses, anal fistulas, perianal infections, diverticular diseases, colitis, and diarrhea.

In certain embodiments, the present invention provides methods for treating, ameliorating and/or preventing gastrointestinal conditions related to SARS-CoV-2 infection (e.g., COVID-19) in a subject, comprising administering to the subject a pharmaceutical composition comprising lactoferrin. In some embodiments, the pharmaceutical composition comprising lactoferrin is configured for oral administration. In some embodiments, the subject is a human subject. In some embodiments, the subject is a human subject suffering from or at risk of suffering from a condition related to SARS-CoV-2 infection (e.g., COVID-19). In some embodiments, the subject is a human subject suffering from a SARS-CoV-2 viral infection. In some embodiments, the gastrointestinal condition is diarrhea. In some embodiments, the gastrointestinal condition is selected from constipation, irritable bowel syndrome, hemorrhoids, anal fissures, perianal abscesses, anal fistulas, perianal infections, diverticular diseases, colitis, and diarrhea.

In certain embodiments, the present invention provides methods for inhibiting viral entry in a cell, comprising exposing the cell a pharmaceutical composition comprising lactoferrin. In some embodiments, the cell is at risk of viral infection (e.g., a cell at risk of SARS-CoV-2 infection). In some embodiments, the cell has been exposed to a virus (e.g., a cell currently exposed to SARS-CoV-2). In some embodiments, the cell is in culture. In some embodiments, the cell is a living cell in a subject (e.g., a human subject) (e.g., a human subject suffering from COVID-19) (e.g., a human subject at risk of suffering from COVID-19). In some embodiments, exposure of the cell to the pharmaceutical composition comprising lactoferrin results in suppression of pro-inflammatory cytokine activity (e.g., IL-6 activity) within the cell. In some embodiments, exposure of the cell to the pharmaceutical composition comprising lactoferrin results in enhancement of NK cell activity within the cell. In some embodiments, exposure of the cell to the pharmaceutical composition comprising lactoferrin results in enhancement of neutrophil activity within the cell. In some embodiments, exposure of the cell to the pharmaceutical composition comprising lactoferrin results in inhibition of viral entry into the cell through inhibiting binding of the virus with heparin sulfate proteoglycan within the cell.

In certain embodiments, the present invention provides methods for treating, ameliorating and/or preventing a condition related to viral infection in a subject, comprising administering to the subject a pharmaceutical composition comprising an IV formulation of recombinant lactoferrin and remdesivir. In some embodiments, the subject is a human subject. In some embodiments, the subject is a human subject suffering from or at risk of suffering from a condition related to SARS-CoV-2 infection (e.g., COVID-19). In some embodiments, the viral infection is a SARS-CoV-2 viral infection.

In certain embodiments, the present invention provides methods for treating, ameliorating and/or preventing SARS-CoV-2 infection (e.g., COVID-19) in a subject, comprising administering to the subject a pharmaceutical composition comprising an IV formulation of recombinant lactoferrin and remdesivir. In some embodiments, the subject is a human subject.

In certain embodiments, the present invention provides methods for treating, ameliorating and/or preventing symptoms related to viral infection in a subject, comprising administering to the subject a pharmaceutical composition comprising an IV formulation of recombinant lactoferrin and remdesivir. In some embodiments, the subject is a human subject. In some embodiments, the subject is a human subject suffering from or at risk of suffering from a condition related to SARS-CoV-2 infection (e.g., COVID-19). In some embodiments, the subject is a human subject suffering from a SARS-CoV-2 viral infection. In some embodiments, the one or more symptoms related to viral infection includes, but is not limited to, fever, fatigue, dry cough, myalgias, dyspnea, acute respiratory distress syndrome, and pneumonia.

In certain embodiments, the present invention provides methods for treating, ameliorating and/or preventing symptoms related to SARS-CoV-2 infection (e.g., COVID-19) in a subject, comprising administering to the subject a pharmaceutical composition comprising an IV formulation of recombinant lactoferrin and remdesivir. In some embodiments, the subject is a human subject. In some embodiments, the one or more symptoms related to viral infection includes, but is not limited to, fever, fatigue, dry cough, myalgias, dyspnea, acute respiratory distress syndrome, and pneumonia.

In certain embodiments, the present invention provides methods for treating, ameliorating and/or preventing acute respiratory distress syndrome in a subject, comprising administering to the subject a pharmaceutical composition comprising an IV formulation of recombinant lactoferrin and remdesivir. In some embodiments, the subject is a human subject. In some embodiments, the subject is a human subject suffering from or at risk of suffering from a condition related to SARS-CoV-2 infection (e.g., COVID-19). In some embodiments, the subject is a human subject suffering from a SARS-CoV-2 viral infection.

In certain embodiments, the present invention provides methods for treating, ameliorating and/or preventing acute respiratory distress syndrome related to SARS-CoV-2 infection (e.g., COVID-19) in a subject, comprising administering to the subject a pharmaceutical composition comprising an IV formulation of recombinant lactoferrin and remdesivir. In some embodiments, the subject is a human subject. In some embodiments, the subject is a human subject suffering from or at risk of suffering from a condition related to SARS-CoV-2 infection (e.g., COVID-19). In some embodiments, the subject is a human subject suffering from a SARS-CoV-2 viral infection.

In certain embodiments, the present invention provides methods for treating, ameliorating and/or preventing pneumonia in a subject, comprising administering to the subject a pharmaceutical composition comprising an IV formulation of recombinant lactoferrin and remdesivir. In some embodiments, the subject is a human subject. In some embodiments, the subject is a human subject suffering from or at risk of suffering from a condition related to SARS-CoV-2 infection (e.g., COVID-19). In some embodiments, the subject is a human subject suffering from a SARS-CoV-2 viral infection.

In certain embodiments, the present invention provides methods for treating, ameliorating and/or preventing pneumonia related to SARS-CoV-2 infection (e.g., COVID-19) in a subject, comprising administering to the subject a pharmaceutical composition comprising an IV formulation of recombinant lactoferrin and remdesivir. In some embodiments, the subject is a human subject. In some embodiments, the subject is a human subject suffering from or at risk of suffering from a condition related to SARS-CoV-2 infection (e.g., COVID-19). In some embodiments, the subject is a human subject suffering from a SARS-CoV-2 viral infection.

In some embodiments involving the treatment of acute respiratory distress syndrome and/or pneumoina, the pharmaceutical composition comprising an IV formulation of recombinant lactoferrin and remdesivir is administered in combination with a known agent to treat respiratory diseases. Known or standard agents or therapies that are used to treat respiratory diseases include, anti-asthma agent/therapies, anti-rhinitis agents/therapies, anti-sinusitis agents/therapies, anti-emphysema agents/therapies, anti-bronchitis agents/therapies or anti-chronic obstructive pulmonary disease agents/therapies. Anti-asthma agents/therapies include mast cell degranulation agents, leukotriene inhibitors, corticosteroids, beta-antagonists, IgE binding inhibitors, anti-CD23 antibody, tryptase inhibitors, and VIP agonists. Anti-allergic rhinitis agents/therapies include H1 antihistamines, alpha-adrenergic agents, and glucocorticoids. Anti-chronic sinusitis therapies include, but are not limited to surgery, corticosteroids, antibiotics, anti-fungal agents, salt-water nasal washes or sprays, anti-inflammatory agents, decongestants, guaifensesin, potassium iodide, luekotriene inhibitors, mast cell degranulating agents, topical moisterizing agents, hot air inhalation, mechanical breathing devices, enzymatic cleaners and antihistamine sprays. Anti-emphysema, anti-bronchitis or anti-chronic obstructive pulmonary disease agents/therapies include, but are not limited to oxygen, bronchodilator agents, mycolytic agents, steroids, antibiotics, anti-fungals, moisterization by nebulization, anti-tussives, respiratory stimulants, surgery and alpha 1 antitrypsin.

In certain embodiments, the present invention provides methods for treating, ameliorating and/or preventing gastrointestinal conditions in a subject, comprising administering to the subject a pharmaceutical composition comprising an IV formulation of recombinant lactoferrin and remdesivir. In some embodiments, the subject is a human subject. In some embodiments, the subject is a human subject suffering from or at risk of suffering from a condition related to SARS-CoV-2 infection (e.g., COVID-19). In some embodiments, the subject is a human subject suffering from a SARS-CoV-2 viral infection. In some embodiments, the gastrointestinal condition is diarrhea. In some embodiments, the gastrointestinal condition is selected from constipation, irritable bowel syndrome, hemorrhoids, anal fissures, perianal abscesses, anal fistulas, perianal infections, diverticular diseases, colitis, and diarrhea.

In certain embodiments, the present invention provides methods for treating, ameliorating and/or preventing gastrointestinal conditions related to SARS-CoV-2 infection (e.g., COVID-19) in a subject, comprising administering to the subject a pharmaceutical composition comprising an IV formulation of recombinant lactoferrin and remdesivir. In some embodiments, the subject is a human subject. In some embodiments, the subject is a human subject suffering from or at risk of suffering from a condition related to SARS-CoV-2 infection (e.g., COVID-19). In some embodiments, the subject is a human subject suffering from a SARS-CoV-2 viral infection. In some embodiments, the gastrointestinal condition is diarrhea. In some embodiments, the gastrointestinal condition is selected from constipation, irritable bowel syndrome, hemorrhoids, anal fissures, perianal abscesses, anal fistulas, perianal infections, diverticular diseases, colitis, and diarrhea.

In certain embodiments, the present invention provides methods for inhibiting viral entry in a cell, comprising exposing the cell a pharmaceutical composition comprising an IV formulation of recombinant lactoferrin and remdesivir. In some embodiments, the cell is at risk of viral infection (e.g., a cell at risk of SARS-CoV-2 infection). In some embodiments, the cell has been exposed to a virus (e.g., a cell currently exposed to SARS-CoV-2). In some embodiments, the cell is in culture. In some embodiments, the cell is a living cell in a subject (e.g., a human subject) (e.g., a human subject suffering from COVID-19) (e.g., a human subject at risk of suffering from COVID-19). In some embodiments, exposure of the cell to the pharmaceutical composition comprising an IV formulation of recombinant lactoferrin and remdesivir results in suppression of pro-inflammatory cytokine activity (e.g., IL-6 activity) within the cell. In some embodiments, exposure of the cell to the pharmaceutical composition comprising an IV formulation of recombinant lactoferrin and remdesivir results in enhancement of NK cell activity within the cell. In some embodiments, exposure of the cell to the pharmaceutical composition comprising an IV formulation of recombinant lactoferrin and remdesivir results in enhancement of neutrophil activity within the cell. In some embodiments, exposure of the cell to the pharmaceutical composition comprising an IV formulation of recombinant lactoferrin and remdesivir results in inhibition of viral entry into the cell through inhibiting binding of the virus with heparin sulfate proteoglycan within the cell.

In certain embodiments, the present invention provides kits comprising (1) a pharmaceutical composition comprising an IV formulation of recombinant lactoferrin and remdesivir, (2) a container, pack, or dispenser, and (3) instructions for administration.

In certain embodiments, the present invention provides kits comprising (1) a pharmaceutical composition comprising recombinant lactoferrin, (2) a container, pack, or dispenser, and (3) instructions for administration. In some emodidments, the kit further comprises remdesivir.

In certain embodiments, the present invention provides kits comprising (1) a pharmaceutical composition comprising lactoferrin, (2) a container, pack, or dispenser, and (3) instructions for administration. In some emodidments, the kit further comprises remdesivir.

In certain embodiments, the present invention provides kits comprising a pharmaceutical composition comprising lactoferrin (e.g., naturally occurring lactoferrin, recombinant lactoferrin) (e.g., oral formulation of lactoferrin, intravenous (IV) formulation of lactoferrin) alone or with additional agents (e.g., remdesivir), and one or more of (1) a container, pack, or dispenser, (2) one or more additional agents (e.g., hydroxychloroquine, dexamethasone, and remdesivir), and (3) instructions for administration

### BRIEF DESCRIPTION OF THE DRAWINGS

FIG. 1A-B: SARS-CoV-2 infectivity high content imaging bioassay showing Huh7 cells with nuclei (blue), viral nucleocapsid protein (red) and neutral lipid droplets (green). A) Vehicle controls showing clusters of red infected cells (syncytia). B) Cells treated with 50 nanomolar Remdesivir, a viral replication inhibitor. Small red spots indicate aborted viral replication.

FIG. 2A-B: SARS-CoV-2 infectivity high content imaging bioassay showing Huh7 cells with nuclei (blue) and viral nucleocapsid protein (red). A) Vehicle controls showing clusters of red infected cells (syncytia). B) Cells treated with lactoferrin.

FIG. 3A-B: Dose response curves for lactoferrin showing A.) reduction of SARS-CoV-2 syncytia formation compared to viral control and B.) resulting inhibition of SARS-CoV-2 infection as represented by 100% efficacy at all concentrations of lactoferrin tested.

FIG. 4: Antiviral activity of a combination of remdesivir and lactoferrin versus remdesivir alone.

### DETAILED DESCRIPTION OF THE INVENTION

A novel coronavirus SARS-CoV-2, also called novel coronavirus 2019 (nCoV-19), started to circulate among humans around December 2019, and it is now widespread as a global pandemic. The disease caused by SARS-CoV-2 virus is called COVID-19, which is highly contagious and has an overall mortality rate of 4.5% as of Mar. 26, 2020. There is no vaccine or antiviral available for SARS-CoV-2.

Experiments implemented during the course of developing embodiments for the present invention conducted a high throughput screen of FDA-approved drugs for efficacy in a SARS-CoV-2 infectivity assay. This high content bioimaging-based assay has proven to be highly robust and informative and has already led to the identification of FDA approved drugs that can be rapidly translated into clinical use. This high-content screen identifies individual agents effective in reducing viral infectivity or replication by imaging individual SARS Cov-2 infected cells. However, because these drugs have not been optimized for anti-Covid19 efficacy, it was hypothesized that combining synergistic drugs representing multiple mechanisms of action into antiviral cocktails could deliver a highly efficacious therapy for Covid19. This strategy has proven highly effective in other viral diseases like HIV and Hepatitis C, where 3 or 4 drug cocktails result in remission of symptoms and undetectable viral load in the patient. Candidate cocktails are evaluated in this high throughput assay with the goal of detecting synergistic efficacy.

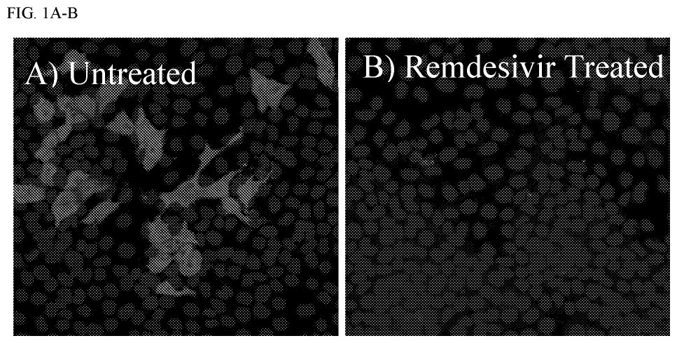

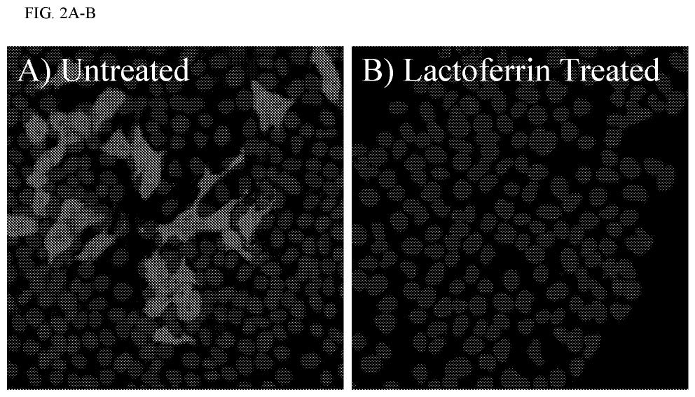

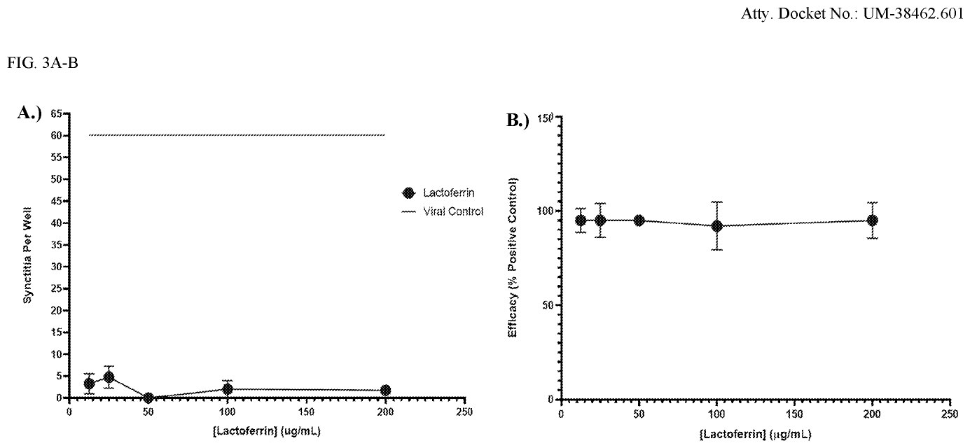

Such experiments resulted in a completed screening of 1,200 compounds through the validated assay platform and resulted in the selection of Remdesivir, Gilead's viral replication inhibitor, as a positive control which resulted in 100% efficacy (see, FIG. 1). Such experiments further demonstrated that lactoferrin showed nearly 100% efficacy in preventing SARS-CoV-2 infection in Huh-7 cells as shown in FIG. 2. Moreover, a dose response study of lactoferrin showed that there was over a 95% reduction in SARS-Cov-2 synctitia at all concentrations tested ranging from 200 ug/mL to 12.5 ug/mL. The characteristic reduction is synctitia, as well as the efficacy of lactoferrin are shown in FIG. 3.

Accordingly, the present invention relates to pharmaceutical compositions comprising lactoferrin (e.g., naturally occurring lactoferrin, recombinant lactoferrin) which function as inhibitors of severe acute respiratory syndrome coronavirus 2 (SARS-CoV-2) viral entry into a subject (e.g., human subject). The invention further relates to methods of preventing, treating and/or ameliorating symptoms related to conditions caused by the SARS-CoV-2 virus (e.g., COVID-19), comprising administering to a subject (e.g., human subject) lactoferrin (e.g., naturally occurring lactoferrin, recombinant lactoferrin) (e.g., oral formulation of lactoferrin, intravenous (IV) formulation of lactoferrin) alone or with additional agents (e.g., remdesivir).

An important aspect of the present invention is that the pharmaceutical compositions comprising lactoferrin (e.g., naturally occurring lactoferrin, recombinant lactoferrin) (e.g., oral formulation of lactoferrin, intravenous (IV) formulation of lactoferrin) alone or with additional agents (e.g., remdesivir) are useful in treating viral infection (e.g., SARS-CoV-2 infection) and symtoms related to such a viral infection (e.g., fever, fatigue, dry cough, myalgias, dyspnea, acute respiratory distress syndrome, and pneumonia).

Some embodiments of the present invention provide methods for administering an effective amount of a pharmaceutical composition comprising lactoferrin (e.g., naturally occurring lactoferrin, recombinant lactoferrin) (e.g., oral formulation of lactoferrin, intravenous (IV) formulation of lactoferrin) alone or with additional agents (e.g., remdesivir) and at least one additional therapeutic agent (including, but not limited to, any pharmaceutical agent useful in treating SARS-CoV-2 infection and/or symtoms related to such a viral infection (e.g., fever, fatigue, dry cough, myalgias, dyspnea, acute respiratory distress syndrome, and pneumonia).

The pharmaceutical compositions of the invention may be administered to any patient that may experience the beneficial effects of lactoferrin. Foremost among such patients are mammals, e.g., humans, although the invention is not intended to be so limited. Other patients include veterinary animals (cows, sheep, pigs, horses, dogs, cats and the like).

The lactoferrin and pharmaceutical compositions may be administered by any means that achieve their intended purpose. For example, administration may be by parenteral, subcutaneous, intravenous, intramuscular, intraperitoneal, transdermal, buccal, intrathecal, intracranial, intranasal or topical routes. Alternatively, or concurrently, administration may be by the oral route. The dosage administered will be dependent upon the age, health, and weight of the recipient, kind of concurrent treatment, if any, frequency of treatment, and the nature of the effect desired.

The pharmaceutical preparations of the present invention are manufactured in a manner that is itself known, for example, by means of conventional mixing, granulating, dragee-making, dissolving, or lyophilizing processes. Thus, pharmaceutical preparations for oral use can be obtained by combining the active mimetic peptides with solid excipients, optionally grinding the resulting mixture and processing the mixture of granules, after adding suitable auxiliaries, if desired or necessary, to obtain tablets or dragee cores.

Suitable excipients are, in particular, fillers such as saccharides, for example lactose or sucrose, mannitol or sorbitol, cellulose preparations and/or calcium phosphates, for example tricalcium phosphate or calcium hydrogen phosphate, as well as binders such as starch paste, using, for example, maize starch, wheat starch, rice starch, potato starch, gelatin, tragacanth, methyl cellulose, hydroxypropylmethylcellulose, sodium carboxymethylcellulose, and/or polyvinyl pyrrolidone. If desired, disintegrating agents may be added such as the above-mentioned starches and also carboxymethyl-starch, cross-linked polyvinyl pyrrolidone, agar, or alginic acid or a salt thereof, such as sodium alginate. Auxiliaries are, above all, flow-regulating agents and lubricants, for example, silica, talc, stearic acid or salts thereof, such as magnesium stearate or calcium stearate, and/or polyethylene glycol. Dragee cores are provided with suitable coatings which, if desired, are resistant to gastric juices. For this purpose, concentrated saccharide solutions may be used, which may optionally contain gum arabic, talc, polyvinyl pyrrolidone, polyethylene glycol and/or titanium dioxide, lacquer solutions and suitable organic solvents or solvent mixtures. In order to produce coatings resistant to gastric juices, solutions of suitable cellulose preparations such as acetylcellulose phthalate or hydroxypropylmethyl-cellulose phthalate, are used. Dye-stuffs or pigments may be added to the tablets or dragee coatings, for example, for identification or in order to characterize combinations of active mimetic peptide doses.

Suitable formulations for parenteral administration include aqueous solutions of the active mimetic peptides in water-soluble form, for example, water-soluble salts and alkaline solutions. In addition, suspensions of the active mimetic peptides as appropriate oily injection suspensions may be administered. Suitable lipophilic solvents or vehicles include fatty oils, for example, sesame oil, or synthetic fatty acid esters, for example, ethyl oleate or triglycerides or polyethylene glycol-400. Aqueous injection suspensions may contain substances which increase the viscosity of the suspension include, for example, sodium carboxymethyl cellulose, sorbitol, and/or dextran. Optionally, the suspension may also contain stabilizers.

One of ordinary skill in the art will readily recognize that the foregoing represents merely a detailed description of certain preferred embodiments of the present invention. Various modifications and alterations of the compositions and methods described above can readily be achieved using expertise available in the art and are within the scope of the invention.

### Experimental

### Example I

Experiments implemented during the course of developing embodiments for the present invention conducted a high throughput screen of FDA-approved drugs for efficacy in a SARS-CoV-2 infectivity assay. This high content bioimaging-based assay has proven to be highly robust and informative and has already led to the identification of FDA approved drugs that can be rapidly translated into clinical use. This high-content screen identifies individual agents effective in reducing viral infectivity or replication by imaging individual SARS Cov-2 infected cells. However, because these drugs have not been optimized for anti-Covid19 efficacy, it was hypothesized that combining synergistic drugs representing multiple mechanisms of action into antiviral cocktails could deliver a highly efficacious therapy for Covid19. This strategy has proven highly effective in other viral diseases like HIV and Hepatitis C, where 3 or 4 drug cocktails result in remission of symptoms and undetectable viral load in the patient. Candidate cocktails are evaluated in this high throughput assay with the goal of detecting synergistic efficacy.

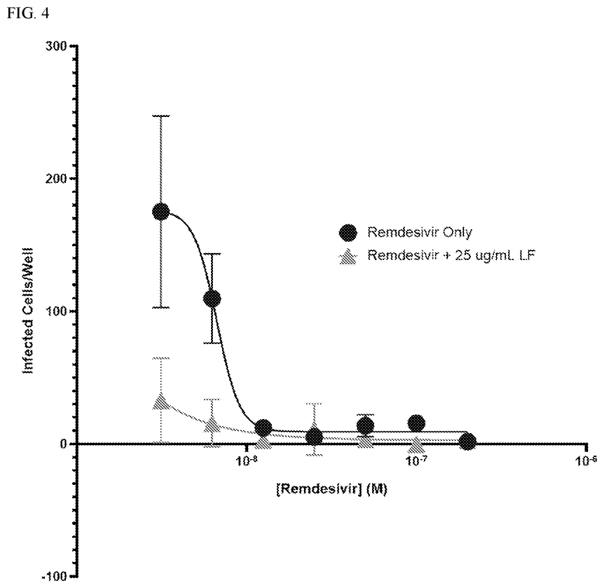

Such experiments resulted in a completed screening of 1,200 compounds through the validated assay platform and resulted in the selection of Remdesivir, Gilead's viral replication inhibitor, as a positive control which resulted in 100% efficacy (see, FIG. 1). Such experiments further demonstrated that lactoferrin showed nearly 100% efficacy in preventing SARS-CoV-2 infection in Huh-7 cells as shown in FIG. 2. Moreover, a dose response study of lactoferrin showed that there was over a 95% reduction in SARS-Cov-2 synctitia at all concentrations tested ranging from 200 ug/mL to 12.5 ug/mL. The characteristic reduction is synctitia, as well as the efficacy of lactoferrin are shown in FIG. 3. FIG. 4 shows the amount of infected cells upon exposure to a combination of remesivir and lactoferrin in comparison to only remdesivir. As shown, the combination of remdesivir and lactoferrin demonstrated increased anti-viral activity in comparison to only administering remdesivir.

Having now fully described the invention, it will be understood by those of skill in the art that the same can be performed within a wide and equivalent range of conditions, formulations, and other parameters without affecting the scope of the invention or any embodiment thereof. All patents, patent applications and publications cited herein are fully incorporated by reference herein in their entirety.

### Equivalents

The invention may be embodied in other specific forms without departing from the spirit or essential characteristics thereof. The foregoing embodiments are therefore to be considered in all respects illustrative rather than limiting the invention described herein. Scope of the invention is thus indicated by the appended claims rather than by the foregoing description, and all changes that come within the meaning and range of equivalency of the claims are intended to be embraced therein.

### Incorporation by Reference

The entire disclosure of each of the patent documents and scientific articles referred to herein is incorporated by reference for all purposes.

What is claimed is:

1. A pharmaceutical composition comprising lactoferrin, wherein the lactoferrin is naturally occurring lactoferrin or recombinant lactoferrin, wherein the lactoferrin is an oral formulation of lactoferrin or an intravenous (IV) formulation of lactoferrin.

2. The pharmaceutical composition of claim 1, wherein the pharmaceutical composition comprising lactoferrin is configured for dispersion in a pharmaceutically acceptable carrier.

3. The method of claim 1, wherein the recombinant lactoferrin is recombinant mammalian lactoferrin.

4. The method of claim 3, wherein the recombinant mammalian lactoferrin is recomibant human lactoferrin.

5. The method of claim 3, wherein the recombinant mammalian lactoferrin is recombiant bovine lactoferrin.

6. The method of claim 1, wherein the lactoferrin is naturally occurring mammalian lactoferrin (e.g., human or bovine).

7. A method for treating, ameliorating and/or preventing a condition related to viral infection in a subject, comprising administering to the subject the pharmaceutical composition recited in claim 1 alone or one or more additional agents.

8. The method of claim 7, wherein the condition related to viral infection is SARS-CoV-2 infection (e.g., COVID-19).

9. The method of claim 7, wherein the subject is a human subject suffering from or at risk of suffering from a condition related to SARS-CoV-2 infection (e.g., COVID-19).

10. The method of claim 7, wherein the pharmaceutical composition comprising lactoferrin is dispersed in a pharmaceutically acceptable carrier.

11. The method of claim 7, wherein the administering is intravenous administration or oral administration.

12. The method of claim 7, wherein the amount of the pharmaceutical composition that is administered is about 1 mg to about 10 g per day.

13. The method of claim 7, wherein the amount of the pharmaceutical composition that is administered is about 10 mg to about 1 g per day.

14. The method of claim 7, further comprising administering to the subject an anti-viral agent.

15. The method of claim 7, wherein the one or more additional agents are selected from hydroxychloroquine, dexamethasone, and remdesivir.

16. The method of claim 7, wherein administration of the pharmaceutical composition results in one or more of suppression of pro-inflammatory cytokine activity (e.g., IL-6 activity) within the subject, enhancement of NK cell activity within the subject, enhancement of neutrophil activity within the subject, and inhibition of viral entry into the subject's cells through inhibiting binding of the virus with heparin sulfate proteoglycan within such cells.

17. The method of claim 7, wherein the one or more additional is remdesivir.

18. A method for treating, ameliorating and/or preventing SARS-CoV-2 infection (e.g., COVID-19) in a subject, comprising administering to the subject the pharmaceutical composition recited in claim 1 alone or with one or more additional agents.

19. The method of claim 18, wherein the subject is a human subject suffering from or at risk of suffering from a condition related to SARS-CoV-2 infection (e.g., COVID-19).

20. The method of claim 18, wherein the pharmaceutical composition is dispersed in a pharmaceutically acceptable carrier.

21. The method of claim 18, wherein the administering is intravenous administration.

22. The method of claim 18, wherein the amount of the pharmaceutical composition that is administered is about 1 mg to about 10 g per day.

23. The method of claim 18, wherein the amount of the pharmaceutical composition that is administered is about 10 mg to about 1 g per day.

24. The method of claim 18, further comprising administering to the subject an anti-viral agent.

25. The method of claim 18, wherein administration of the pharmaceutical composition comprising an IV formulation of recombinant lactoferrin and remdesivir results in one or more of suppression of pro-inflammatory cytokine activity (e.g., IL-6 activity) within the subject, enhancement of NK cell activity within the subject, enhancement of neutrophil activity within the subject, and inhibition of viral entry into the subject's cells through inhibiting binding of the virus with heparin sulfate proteoglycan within such cells.

26. The method of claim 18, wherein the one or more additional agents are selected from hydroxychloroquine, dexamethasone, and remdesivir.

27. A method for treating, ameliorating and/or preventing symptoms related to viral infection in a subject, comprising administering to the subject the pharmaceutical composition recited in claim 1 alone or with one or more additional agents.

28. The method of claim 27, wherein the symptoms related to viral infection in a subject are one or more of fever, fatigue, dry cough, myalgias, dyspnea, acute respiratory distress syndrome, and pneumonia.

29. The method of claim 27, wherein the subject is a human subject suffering from or at risk of suffering from a condition related to SARS-CoV-2 infection (e.g., COVID-19).

30. The method of claim 27, wherein the pharmaceutical composition is dispersed in a pharmaceutically acceptable carrier.

31. The method of claim 27, wherein the administering is intravenous administration.

32. The method of claim 27, wherein the amount of the pharmaceutical composition that is administered is about 1 mg to about 10 g per day.

33. The method of claim 27, wherein the amount of the pharmaceutical composition that is administered is about 10 mg to about 1 g per day.

34. The method of claim 27, further comprising administering to the subject an anti-viral agent.

35. The method of claim 27, wherein administration of the pharmaceutical composition comprising an IV formulation of recombinant lactoferrin and remdesivir results in one or more of suppression of pro-inflammatory cytokine activity (e.g., IL-6 activity) within the subject, enhancement of NK cell activity within the subject, enhancement of neutrophil activity within the subject, and inhibition of viral entry into the subject's cells through inhibiting binding of the virus with heparin sulfate proteoglycan within such cells.

36. The method of claim 27, wherein the one or more additional agents are selected from hydroxychloroquine, dexamethasone, and remdesivir.

37. The method of claim 27, wherein the one or more additional agents is remdesivir.

38. A method for treating, ameliorating and/or preventing symptoms related to SARS-CoV-2 infection (e.g., COVID-19) in a subject, comprising administering to the subject a pharmaceutical composition as recited in claim 1 alone or with one or more additional agents.

39. The method of claim 38, wherein the symptoms related to viral infection in a subject are one or more of fever, fatigue, dry cough, myalgias, dyspnea, acute respiratory distress syndrome, and pneumonia.

40. The method of claim 38, wherein the pharmaceutical composition is dispersed in a pharmaceutically acceptable carrier.

41. The method of claim 38, wherein the administering is intravenous administration.

42. The method of claim 38, wherein the amount of the pharmaceutical composition that is administered is about 1 mg to about 10 g per day.

43. The method of claim 38, wherein the amount of the pharmaceutical composition that is administered is about 10 mg to about 1 g per day.

44. The method of claim 38, further comprising administering to the subject an anti-viral agent.

45. The method of claim 38, wherein administration of the pharmaceutical composition comprising an IV formulation of recombinant lactoferrin and remdesivir results in one or more of suppression of pro-inflammatory cytokine activity (e.g., IL-6 activity) within the subject, enhancement of NK cell activity within the subject, enhancement of neutrophil activity within the subject, and inhibition of viral entry into the subject's cells through inhibiting binding of the virus with heparin sulfate proteoglycan within such cells.

46. The method of claim 38, wherein the one or more additional agents are selected from hydroxychloroquine, dexamethasone, and remdesivir.

47. A method for treating, ameliorating and/or preventing acute respiratory distress syndrome in a subject, comprising administering to the subject the pharmaceutical composition recited in claim 1 alone or with one or more additional agents.

48. The method of claim 47, wherein the acute respiratory distress syndrome is related to SARS-CoV-2 infection (e.g., COVID-19).

49. The method of claim 47, wherein the subject is a human subject suffering from or at risk of suffering from a condition related to SARS-CoV-2 infection (e.g., COVID-19).

50. The method of claim 47, wherein the pharmaceutical composition is dispersed in a pharmaceutically acceptable carrier.

51. The method of claim 47, wherein the administering is intravenous administration.

52. The method of claim 47, wherein the amount of the pharmaceutical composition that is administered is about 1 mg to about 10 g per day.

53. The method of claim 47, wherein the amount of the pharmaceutical composition that is administered is about 10 mg to about 1 g per day.

54. The method of claim 47, further comprising administering to the subject an anti-viral agent.

55. The method of claim 47, wherein administration of the pharmaceutical composition comprising an IV formulation of recombinant lactoferrin and remdesivir results in one or more of suppression of pro-inflammatory cytokine activity (e.g., IL-6 activity) within the subject, enhancement of NK cell activity within the subject, enhancement of neutrophil activity within the subject, and inhibition of viral entry into the subject's cells through inhibiting binding of the virus with heparin sulfate proteoglycan within such cells.

56. The method of claim 47, wherein the one or more additional agents are selected from hydroxychloroquine, dexamethasone, and remdesivir.

57. A method for treating, ameliorating and/or preventing acute respiratory distress syndrome related to SARS-CoV-2 infection (e.g., COVID-19) in a subject, comprising administering to the subject the pharmaceutical composition recited in claim 1 alone or with one or more additional agents.

58. The method of claim 57, wherein the subject is a human subject suffering from or at risk of suffering from a condition related to SARS-CoV-2 infection (e.g., COVID-19).

59. The method of claim 57, wherein the pharmaceutical composition is dispersed in a pharmaceutically acceptable carrier.

60. The method of claim 57, wherein the administering is intravenous administration.

61. The method of claim 57, wherein the amount of the pharmaceutical composition that is administered is about 1 mg to about 10 g per day.

62. The method of claim 57, wherein the amount of the pharmaceutical composition that is administered is about 10 mg to about 1 g per day.

63. The method of claim 57, further comprising administering to the subject an anti-viral agent.

64. The method of claim 57, wherein administration of the pharmaceutical composition comprising an IV formulation of recombinant lactoferrin and remdesivir results in one or more of suppression of pro-inflammatory cytokine activity (e.g., IL-6 activity) within the subject, enhancement of NK cell activity within the subject, enhancement of neutrophil activity within the subject, and inhibition of viral entry into the subject's cells through inhibiting binding of the virus with heparin sulfate proteoglycan within such cells.

65. The method of claim 57, wherein the one or more additional agents are selected from hydroxychloroquine, dexamethasone, and remdesivir.

66. A method for treating, ameliorating and/or preventing pneumonia in a subject, comprising administering to the subject the pharmaceutical recited in claim 1 alone or with one or more additional agents.

67. The method of claim 66, wherein the pneumonia is related to SARS-CoV-2 infection (e.g., COVID-19).

68. The method of claim 66, wherein the subject is a human subject suffering from or at risk of suffering from a condition related to SARS-CoV-2 infection (e.g., COVID-19).

69. The method of claim 66, wherein the pharmaceutical composition is dispersed in a pharmaceutically acceptable carrier.

70. The method of claim 66, wherein the administering is intravenous administration.

71. The method of claim 66, wherein the amount of the pharmaceutical composition that is administered is about 1 mg to about 10 g per day.

72. The method of claim 66, wherein the amount of the pharmaceutical composition that is administered is about 10 mg to about 1 g per day.

73. The mentod of claim 66, further comprising administering an additional agent for treating pneumonia and/or an anti-viral agent.

74. The method of claim 66, wherein administration of the pharmaceutical composition comprising an IV formulation of recombinant lactoferrin and remdesivir results in one or more of suppression of pro-inflammatory cytokine activity (e.g., IL-6 activity) within the subject, enhancement of NK cell activity within the subject, enhancement of neutrophil activity within the subject, and inhibition of viral entry into the subject's cells through inhibiting binding of the virus with heparin sulfate proteoglycan within such cells.

75. The method of claim 66, wherein the one or more additional agents are selected from hydroxychloroquine, dexamethasone, and remdesivir.

76. A method for treating, ameliorating and/or preventing pneumonia related to SARS-CoV-2 infection (e.g., COVID-19) in a subject, comprising administering to the subject the pharmaceutical composition recited in claim 1 alone or with one or more additional agents.

77. The method of claim 76, wherein the subject is a human subject suffering from or at risk of suffering from a condition related to SARS-CoV-2 infection (e.g., COVID-19).

78. The method of claim 76, wherein the pharmaceutical composition is dispersed in a pharmaceutically acceptable carrier.

79. The method of claim 76, wherein the administering is intravenous administration.

80. The method of claim 76, wherein the amount of the pharmaceutical composition that is administered is about 1 mg to about 10 g per day.

81. The method of claim 76, wherein the amount of the pharmaceutical composition that is administered is about 10 mg to about 1 g per day.

82. The method of claim 76, further comprising administering to the subject an anti-viral agent.

83. The method of claim 76, wherein administration of the pharmaceutical composition comprising an IV formulation of recombinant lactoferrin and remdesivir results in one or more of suppression of pro-inflammatory cytokine activity (e.g., IL-6 activity) within the subject, enhancement of NK cell activity within the subject, enhancement of neutrophil activity within the subject, and inhibition of viral entry into the subject's cells through inhibiting binding of the virus with heparin sulfate proteoglycan within such cells.

84. The method of claim 76, wherein the one or more additional agents are selected from hydroxychloroquine, dexamethasone, and remdesivir.

85. A method for treating, ameliorating and/or preventing one or more gastrointestinal conditions in a subject, comprising administering to the subject a pharmaceutical composition comprising an IV formulation of recombinant lactoferrin and remdesivir.

86. The method of claim 85, wherein the one or more gastrointestinal conditions are related to SARS-CoV-2 infection (e.g., COVID-19).

87. The method of claim 85, wherein the subject is a human subject suffering from or at risk of suffering from a condition related to SARS-CoV-2 infection (e.g., COVID-19).

88. The method of claim 85, wherein the pharmaceutical composition comprising is dispersed in a pharmaceutically acceptable carrier.

89. The method of claim 85, wherein the one or more gastrointestinal conditions are selected from constipation, irritable bowel syndrome, hemorrhoids, anal fissures, perianal abscesses, anal fistulas, perianal infections, diverticular diseases, colitis, and diarrhea.

90. The method of claim 85, wherein the amount of the pharmaceutical composition that is administered is about 1 mg to about 10 g per day.

91. The method of claim 85, wherein the amount of the pharmaceutical composition that is administered is about 10 mg to about 1 g per day.

92. The method of claim 85, further comprising administering to the subject an anti-viral agent.

93. The method of claim 85, wherein administration of the pharmaceutical composition comprising an IV formulation of recombinant lactoferrin and remdesivir results in one or more of suppression of pro-inflammatory cytokine activity (e.g., IL-6 activity) within the subject, enhancement of NK cell activity within the subject, enhancement of neutrophil activity within the subject, and inhibition of viral entry into the subject's cells through inhibiting binding of the virus with heparin sulfate proteoglycan within such cells.

94. The method of claim 85, wherein the one or more additional agents are selected from hydroxychloroquine, dexamethasone, and remdesivir.

95. A method for treating, ameliorating and/or preventing pneumonia related to SARS-CoV-2 infection (e.g., COVID-19) in a subject, comprising administering to the subject the pharmaceutical composition recited in claim 1 alone or with one or more additional agents.

96. The method of claim 95, wherein the subject is a human subject suffering from or at risk of suffering from a condition related to SARS-CoV-2 infection (e.g., COVID-19).

97. The method of claim 95, wherein the pharmaceutical composition is dispersed in a pharmaceutically acceptable carrier.

98. The method of claim 95, wherein the one or more gastrointestinal conditions are selected from constipation, irritable bowel syndrome, hemorrhoids, anal fissures, perianal abscesses, anal fistulas, perianal infections, diverticular diseases, colitis, and diarrhea.

99. The method of claim 95, wherein the amount of the pharmaceutical composition that is administered is about 1 mg to about 10 g per day.

100. The method of claim 95, wherein the amount of the pharmaceutical composition that is administered is about 10 mg to about 1 g per day.

101. The method of claim 95, further comprising administering to the subject an anti-viral agent.

102. The method of claim 95, wherein administration of the pharmaceutical composition comprising an IV formulation of recombinant lactoferrin and remdesivir results in one or more of suppression of pro-inflammatory cytokine activity (e.g., IL-6 activity) within the subject, enhancement of NK cell activity within the subject, enhancement of neutrophil activity within the subject, and inhibition of viral entry into the subject's cells through inhibiting binding of the virus with heparin sulfate proteoglycan within such cells.

103. The method of claim 95, wherein the one or more additional agents are selected from hydroxychloroquine, dexamethasone, and remdesivir.

104. A method for inhibiting viral entry in a cell, comprising exposing the cell the pharmaceutical composition recited in claim 1 alone or with one or more additional agents.

105. The method of claim 104, wherein cell is at risk of viral infection (e.g., a cell at risk of SARS-CoV-2 infection).

106. The method of claim 104, wherein the cell has been exposed to a virus (e.g., a cell currently exposed to SARS-CoV-2).

107. The method of claim 104, wherein the cell is in culture.

108. The method of claim 104, wherein the cell is a living cell in a subject (e.g., a human subject) (e.g., a human subject suffering from COVID-19) (e.g., a human subject at risk of suffering from COVID-19).

109. The method of claim 104, wherein exposure of the cell to the pharmaceutical composition comprising an IV formulation of recombinant lactoferrin and remdesivir results in one or more of suppression of pro-inflammatory cytokine activity (e.g., IL-6 activity) within the cell, enhancement of NK cell activity within the cell, enhancement of neutrophil activity within the cell, and inhibition of viral entry into the cell through inhibiting binding of the virus with heparin sulfate proteoglycan within the cell.

110. The method of claim 104, wherein the one or more additional agents are selected from hydroxychloroquine, dexamethasone, and remdesivir.

111. A kit comprising the pharmaceutical composition recitedin claim 1, and one or or more of:
one or more additional agents,
a container, pack,
a dispenser, and
instructions for administration.

112. The kit of claim 111, wherein kit further comprises an anti-viral agent.

113. The kit of claim 111, wherein the one or more additional agents are selected from hydroxychloroquine, dexamethasone, and remdesivir.
